(19) United States
(12) Patent Application Publication
(10) Pub. No.: US 2020/0196849 A1
(43) Pub. Date: Jun. 25, 2020

(54) Sealing device capable of automatically stamping

(71) Applicant: Xianda Renn, Qingdao City (CN)

(72) Inventor: Xianda Renn, Qingdao City (CN)

(21) Appl. No.: 16/808,394

(22) Filed: Mar. 4, 2020

(30) Foreign Application Priority Data
Dec. 13, 2019 (CN) 2019112864407

(51) Int. Cl. A61B 1/06; A61B 1/00; A61B 1/04; A61B 1/24

(52) U.S. Cl. CPC A61B 1/0692; A61B 1/00048; A61B 2090/372; A61B 1/24; A61B 1/04

(57) ABSTRACT

The invention discloses a detector capable of autonomously detecting the oral cavity, which includes a headband. The right end of the headband is fixedly provided with a detector, and the left end surface of the detector is fixed with an eyecup. The headband is worn behind the head. The eyecup is covered around the eyes, a display screen is provided in the headband and on the right side of the eyecup, and a light-passing port is provided between the display screen and the eyecup, and in the light-passing port A light blocking plate is provided, and an opening and closing device is provided on the upper side of the display screen and the light-passing port. The opening and closing device is provided with a lifting plate. The present invention is worn on the head and surrounds the eyes with an eye mask. The VR displays the images detected in the oral cavity. By holding a small camera in the oral cavity, the user can clearly see the situation inside the oral cavity and protect privacy. The device has excellent storage and protection. Automatic storage at the push of a button, extremely easy to use.

### CROSS-REFERENCES TO RELATED APPLICATIONS

The present application claims priority from Chinese application No. 2019110434902 filed on Oct. 30, 2019 which is hereby incorporated by reference in its entirety.

### TECHNICAL FIELD

The invention relates to the technical field of vehicles, in particular to a sunscreen and antifouling ceiling device for vehicle.

### BACKGROUND OF THE INVENTION

In today's society, self-driving travel has become a very common mode of travel. More and more people choose to buy private cars, and self-driving is a convenient and universal transportation mode;

Now that the purchase of private cars is becoming more common, the protection of vehicles has received more attention. When the vehicle is parked outdoors, it will be exposed to sunlight and dust will accumulate on the surface of the vehicle. Due to various adverse conditions of outdoor parking, it will cause damage to the vehicle surface, reduce the service life of the vehicle, and increase the maintenance cost of the vehicle.

### BRIEF SUMMARY OF THE INVENTION

The technical problem to be solved by the present invention is to provide a sun-proof and anti-fouling ceiling device for a vehicle, which can reduce damage to the surface of the vehicle such as sunlight and dust during outdoor parking, and achieve better maintenance of the vehicle.

The present invention is achieved by the following technical solutions.

The sun-proof and anti-fouling ceiling device for a vehicle of the present invention comprises four support blocks fixed to the upper wall of the vehicle roof, and two front-side support blocks are rotatably connected to the front driving pulley shaft and the front driven pulley shaft. The front drive pulley shaft is cooperatively connected with the front drive pulley. The front drive pulley shaft is connected with the front drive bevel gear on the rear side of the front drive pulley. The front drive pulley is meshed with the front timing belt to drive the front synchronization. The belt is fixedly connected to the front moving block. The front wall of the front moving block is fixedly connected to a front clamping block. The front clamping block is clamped to the top folding shed. The top folding shed is folded and fixed on the roof of the vehicle. In the top folding shed shrink box of the wall, the front active pulley shaft is driven to rotate by a motor, the motor is fixedly connected to the rear wall of the front support plate fixedly connected to the upper wall of the roof, and the rear wall of the front support plate is fixedly connected A front folding shed mechanism, the front driven pulley shaft is cooperatively connected with the front driven pulley, the front driven pulley is meshed with the front timing belt, and the front driven pulley shaft is connected with the front driven bevel gear, The front driven bevel gear with the right The driving bevel gear is meshed and driven, the right driving bevel gear is connected to the right driving pulley shaft in a cooperative manner, the right driving pulley shaft is rotatably connected to the front right support block, and the right driving pulley shaft is connected to the right folding shed mechanism. The right driven pulley shaft is rotatably connected to the rear right support block, the right driven pulley shaft is connected to the right folding shed mechanism, and the two rear support blocks are rotated with the rear driving pulley shaft and the rear driven pulley shaft. The rear driving pulley shaft is connected with the rear driving pulley in a cooperative manner, the rear driving pulley shaft is connected with the rear driving bevel gear in a front side of the rear driving pulley, and the rear driving pulley meshes with the rear timing belt, The rear timing belt is fixedly connected to the rear moving block, a rear clamping block is fixedly connected to the front wall of the rear moving block, the rear clamping block is clampingly connected to the top folding shed, and the rear driven pulley wheel shaft The rear driven bevel gear is meshed with the left driven bevel gear on the left, and the left driven bevel gear is mated and connected with the left driven bevel shaft. Posterior left end branch The block is rotationally connected, the left driven pulley wheel shaft is cooperatively connected with the left folding shed mechanism, the right folding shed mechanism is linked with the rear folding shed mechanism through a rear rope, and the rear folding shed mechanism is fixedly connected in front of the rear support plate And located on the rear side of the rear timing belt, the front driving bevel gear meshes with the left driving bevel gear on the left side, the left driving bevel gear is cooperatively connected with the left driving pulley shaft, and the left driving pulley shaft is supported by the front left end The block is rotationally connected, and the left driving pulley shaft is cooperatively connected with the left folding shed mechanism. The left folding shed mechanism is linked with the front folding shed mechanism through a front rope. The motor drives the front active pulley shaft to rotate, thereby The meshing transmission of the front driving bevel gear and the left driving bevel gear drives the left folding shed mechanism to work, so that the front folding shed mechanism is driven by the front rope, and the right folding bevel gear is driven by the meshing transmission of the front driven bevel gear and the right driving bevel gear. Work, thereby driving the rear folding shed mechanism to work through the rear rope.

Further, the left folding shed mechanism includes a left driving pulley that is cooperatively connected with the left driving pulley shaft, the left driving pulley is meshed with the left timing belt, and the left timing belt is fixedly connected with the left moving block. A left clamping block is fixedly connected to the left wall of the left moving block, and the left clamping block is clamped and connected to the left folding shed, and the left folding shed is folded and contracted in a left folding shed shrink box fixed on the left front upper wall of the car The left driven pulley shaft is matched with the left driven pulley, and the left driven pulley is meshed with the left timing belt for transmission.

Further, the front folding shed mechanism includes a front folding shed shrink box fixedly connected to a front support plate, the front folding shed shrink box is provided with a front sliding cavity with an opening facing upward, and the bottom wall of the front folding shed shrink box A front contraction groove communicating with the upper and lower sides is provided, and a front sliding guide is provided on the lower wall of the front sliding cavity. A front spring is fixedly connected to the right end wall of the front sliding cavity, and the left side of the front spring is fixedly connected with the sliding cooperation with the front sliding guide. A front sliding block, a front rope is fixedly connected to the left end of the front sliding block, a front hanging bar is arranged above the front sliding block, the front hanging bar is fixedly connected to a front folding shed, and the other end of the front folding shed is fixed It is connected to the rear wall of the front sliding cavity. A front fixing rod is fixed between the left and right walls of the front sliding cavity. The left and right sides of the front drooping rod are fixedly installed on a winding block, and a winding is wound on the winding block on the right side. The front resetting rope passing through the front fixing rod, the left and right winding blocks are fixedly connected to the front telescopic rod group, the left and right front telescopic rod groups are slidingly connected to the front fixing rod through a connecting plate, and the rear wall of the front support plate is fixedly connected There is The front fixing plate on the left side of the folding shed shrink box, the front fixing plate is fixedly connected with a front rope block at the rear wall, and the front rope string passes through the front left and right rope track in the middle, and the front left and right rope track is left The front, rear, and front rope track are connected to the front and rear sides, and the front and back rope track and the front and left and right rope track are slidably connected to the front track wheel. The front track wheel is fixed to the front track in the front track block. The axles are cooperatively connected, the front rope is fixedly connected to the left connecting rod through the front and rear through-rope rails and the front left and right through-rope rails, and the left connecting rod is fixedly connected to the lower wall of the left moving block.

Further, the right folding shed mechanism includes a right driving pulley that is cooperatively connected with the right driving pulley shaft, the right driving pulley is meshed with the right timing belt, the right timing belt is meshed with the right moving block, and the right A right clamping block is fixedly connected to the right wall of the moving block, and the right clamping block is clamped to the right folding shed, and the right folding shed is folded and contracted in a right folding shed shrink box fixed on the upper wall of the right rear end of the vehicle. The right driven pulley shaft is cooperatively connected with the right driven pulley, and the right driven pulley is meshed with the right timing belt for transmission.

Further, the rear folding shed mechanism includes a rear folding shed shrink box fixedly connected to a rear support plate, the rear folding shed shrink box is provided with a rear sliding cavity with an opening facing upward, and the bottom wall of the rear folding shed shrink box A rear contraction groove communicating with the upper and lower sides is provided. A rear slide rail is provided on the lower wall of the rear slide cavity. A rear spring is fixedly connected to the left end wall of the rear slide cavity. A right side of the rear spring is fixedly connected to the rear slide rail. The rear sliding block with sliding fit is fixedly connected with a rear rope at the right end surface of the rear sliding block. A rear hanging bar is arranged above the rear sliding block. The rear hanging bar is fixedly connected with a rear folding shed. One end is fixedly connected to the rear wall of the rear sliding cavity. Rear fixing rods are fixed between the left and right walls of the rear sliding cavity. The left and right sides of the rear drooping rod are fixedly installed on a winding block, and the left side is wound on the winding block. There is a rear reset rope that crosses the rear fixing rod, the left and right winding blocks are fixedly connected to the rear telescopic rod group, the left and right rear telescopic rod groups are slidingly connected to the rear fixed rod through a connecting plate, and the front wall of the rear support plate is fixed Connected with A rear fixing plate on the right side of the shrink box of the rear folding shed, a rear through rope block is fixedly connected to the front wall of the rear fixing plate, and the left and right through rope tracks penetrate through the middle of the rear through rope block, and the rear left and right rope A rear front and rear rope track communicates with the front and back of the right side of the track. The rear front and rear rope track is slidably connected to the rear left and right rope track at the junction with the rear track wheel and the rear track wheel is fixedly connected to the rear track block. The rear turning wheel axle is cooperatively connected, the rear rope is fixedly connected to the right connecting rod through the rear front and rear through-rail track and the rear left and right through-rail track, and the right connecting rod is fixedly connected to the lower wall of the right moving block.

Further, the front telescopic rod group includes a plurality of front connecting rods, and the plurality of front connecting rods are rotationally connected through a plurality of front circular hinges.

Further, the rear telescopic rod group includes a plurality of rear connecting rods, and the plurality of rear connecting rods are rotationally connected through a plurality of rear circular hinges.

The beneficial effects of the present invention are: the device has a simple structure, convenient operation, and high degree of automation. When the device is in operation, the front drive pulley shaft can be driven by the motor to rotate, so that the left drive is driven by the meshing transmission between the front drive bevel gear and the left drive bevel gear The folding shed mechanism works, so that the front folding shed mechanism is driven by the front rope, and the right driven shed mechanism is driven by the meshing transmission of the front driven bevel gear and the right driving bevel gear, so that the rear folding shed mechanism is driven by the rear rope. Folding sheds are deployed to cover and protect the vehicle surface, reducing the maintenance cost of the vehicle and increasing the service life of the vehicle.

### BRIEF DESCRIPTION OF THE DRAWINGS

In order to more clearly explain the embodiments of the invention or the technical solutions in the prior art, the drawings used in the description of the embodiments or the prior art are briefly introduced below. Obviously, the drawings in the following description are merely For some embodiments of the invention, for those of ordinary skill in the art, other drawings can be obtained based on these drawings without paying creative labor.

FIG. 1 is a schematic structural diagram of an embodiment of the present invention;

2 is a schematic plan view of a structure of an embodiment of the present invention;

3 is a schematic diagram of A-A in FIG. 1 according to an embodiment of the present invention;

FIG. 4 is an enlarged schematic view of the structure at B in FIG. 2 according to the embodiment of the present invention;

FIG. 5 is an enlarged schematic view of the structure at C in FIG. 2 according to the embodiment of the present invention;

6 is a schematic diagram of D-D in FIG. 2 according to an embodiment of the present invention;

7 is a part view of the front telescopic rod group 33 in FIG. 1 according to the embodiment of the present invention;

FIG. 8 is a part view of the rear telescopic rod group 94 in FIG. 1 according to the embodiment of the present invention.

### DETAILED DESCRIPTION OF THE INVENTION

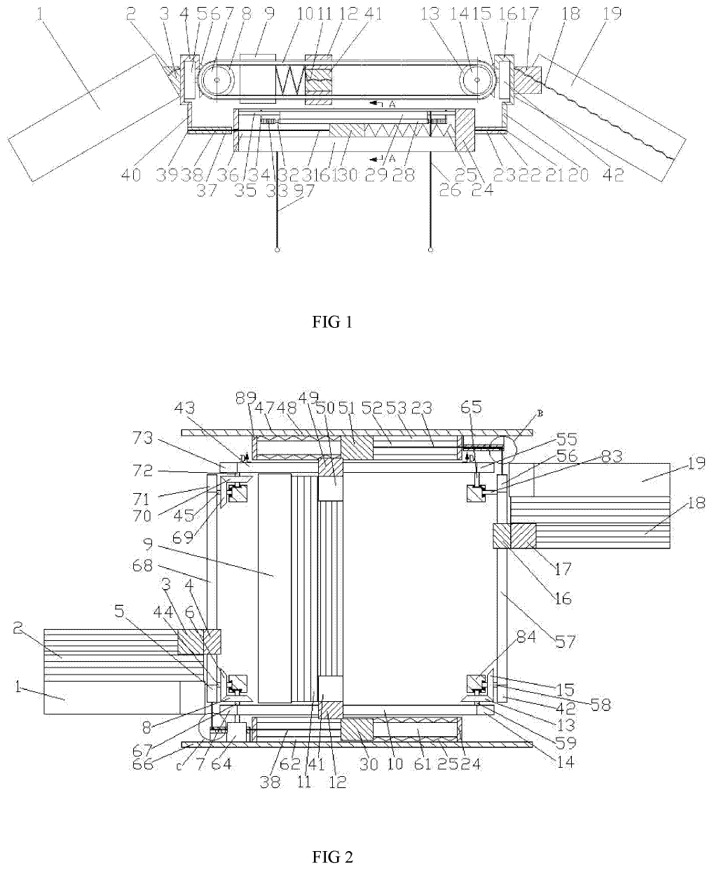

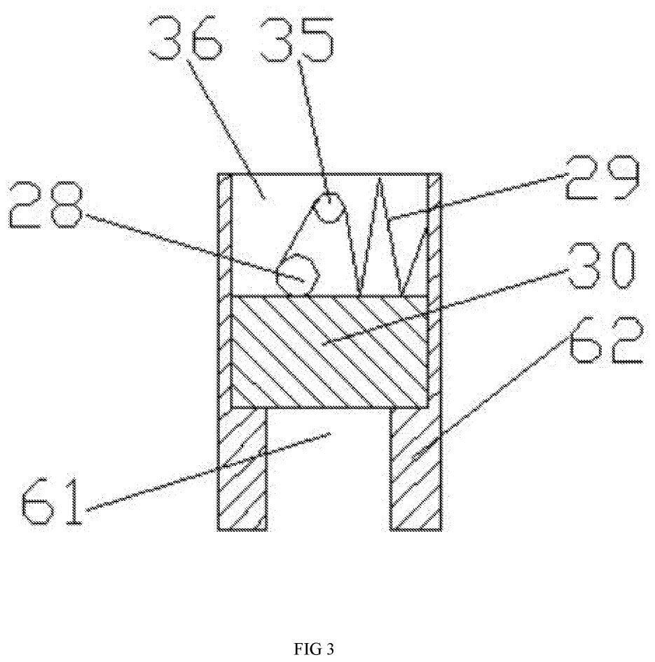

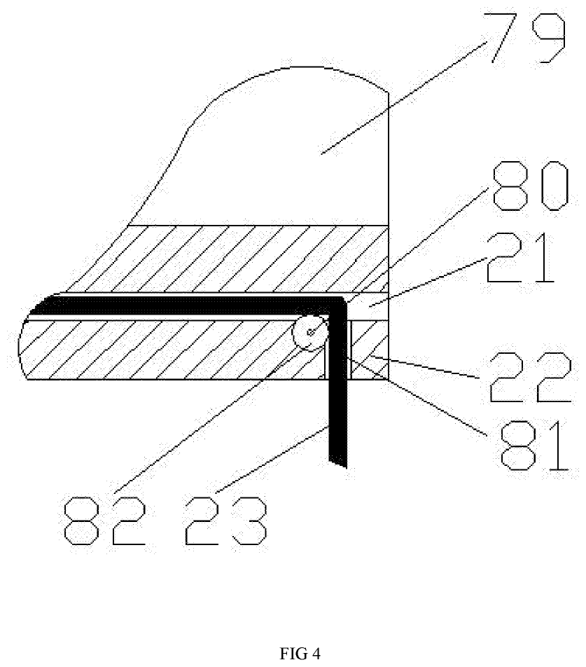

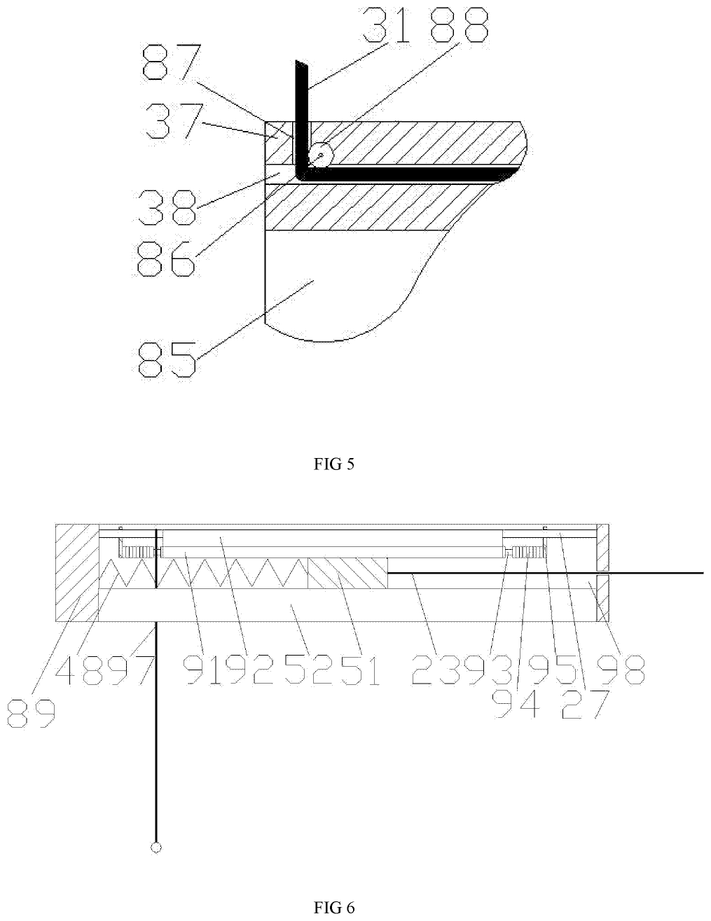

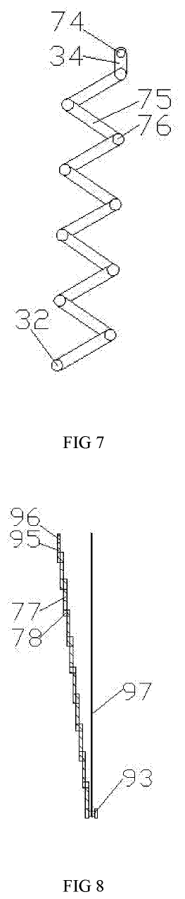

The present invention will be described in detail below with reference to FIGS. 1-8. For convenience of description, the orientation described below is defined as follows: the up-down, left-right, front-back direction described below is consistent with the up-down, left-right, front-back direction of the projection relationship of FIG. 1 itself.

The sunscreen and antifouling ceiling device for vehicles described in conjunction with FIGS. 1-8 includes four support blocks 84 fixed to the upper wall of the roof, two front side support blocks 84 and a front active pulley shaft 67 and a front The driven pulley shaft 59 is rotationally connected, the front driving pulley shaft 67 is cooperatively connected with the front driving pulley 7, and the front driving pulley shaft 67 is cooperatively connected with the front driving bevel gear 8 at the rear side of the front driving pulley 7, The front driving pulley 7 meshes with the front timing belt 10, and the front timing belt 10 is fixedly connected to the front moving block 12. The front moving block 12 is fixedly connected with a front clamping block 41 at the rear wall. 41 is clamped and connected with the top folding shed 11 which is folded and shrunk in the top folding shed shrink box 9 fixed on the upper wall of the vehicle roof. The front driving pulley shaft 67 is driven to rotate by a motor 64. The motor 64 is fixedly connected to the rear wall of a front support plate 66 fixedly connected to the upper wall of the roof. The front support plate 66 is fixedly connected to a front folding shed mechanism. The front driven pulley shaft 59 and the front driven pulley 14 In a cooperative connection, the front driven pulley 14 meshes with the front timing belt 10, and the front driven pulley shaft 59 and The driven bevel gear 13 is mated and connected. The front driven bevel gear 13 meshes with the right driving bevel gear 15 on the right side. The right driving bevel gear 15 is mated with the right driving pulley shaft 58 which is the right driving pulley shaft 58. It is rotatably connected with the front right support block 84, the right driving pulley shaft 58 is cooperatively connected with the right folding shed mechanism, the right driven pulley shaft 83 is rotatably connected with the right rear supporting block 84, and the right driven pulley shaft 83 is cooperatively connected with the right folding shed mechanism, two rear side support blocks 84 are rotationally connected with the rear driving pulley shaft 72 and the rear driven pulley shaft 65, and the rear driving pulley shaft 72 is connected with the rear driving pulley 73, The rear driving pulley shaft 72 is cooperatively connected with the rear driving bevel gear 70 on the front side of the rear driving pulley 73. The rear driving pulley 73 meshes with the rear timing belt 43 and the rear timing belt 43 and the rear moving block 49 The rear moving block 49 is fixedly connected to the front wall of the rear clamping block 50. The rear clamping block 50 is clamped to the top folding shed 11. The rear driven pulley shaft 65 is connected to the rear driven. The pulley 55 is cooperatively connected, and the rear driving bevel gear 70 is connected to the left and left driven cones. Gear 69 meshes with the transmission. The left driven bevel gear 69 is cooperatively connected with the left driven pulley shaft 45. The left driven pulley shaft 45 is rotatably connected with the rear left support block 84. The left driven pulley shaft 45 is connected with The left folding shed mechanism is cooperatively connected, and the right folding shed mechanism is linked with the rear folding shed mechanism through a rear rope 23, which is fixedly connected to the front wall of the rear support plate 47 and located on the rear side of the rear timing belt 43, The front driving bevel gear 8 is meshed with the left driving bevel gear 6 on the left side. The left driving bevel gear 6 is connected to the left driving pulley shaft 44 in a rotational connection with the front left support block 84. The left active pulley shaft 44 is cooperatively connected with the left folding shed mechanism, and the left folding shed mechanism is linked with the front folding shed mechanism through a front rope 38. The motor 64 drives the front active pulley shaft 67 to rotate, thereby The meshing transmission of the front driving bevel gear 8 and the left driving bevel gear 6 drives the operation of the left folding shed mechanism, so that the front folding shed mechanism works through the front rope 38, and the front driven bevel gear 13 and the right driving bevel gear 15 The meshing transmission drives the right folding shed mechanism to work, thereby driving the rear folding shed mechanism through the rear rope 23 to work.

Beneficially, the left folding shed mechanism includes a left driving pulley 5 cooperatively connected with a left driving pulley shaft 44, the left driving pulley 5 meshes with the left timing belt 68, and the left timing belt 68 and the left moving block 4 fixed connection, the left moving block 4 is fixedly connected with the left clamping block 3 on the left wall, the left clamping block 3 is clamped and connected with the left folding shed 2, and the left folding shed 2 is folded and fixed on the left side of the car In the left folding shed contraction box 1 on the upper side of the front end, the left driven pulley shaft 45 cooperates with the left driven pulley 71, and the left driven pulley 71 meshes with the left timing belt 68 for transmission.

Advantageously, the front folding shed mechanism includes a front folding shed shrink box 24 fixedly connected to a front support plate 66, the front folding shed shrink box 24 is provided with a front sliding cavity 36 with an opening facing upward, and the front folding shed shrinks The bottom wall of the box 24 is provided with a front contraction groove 61 communicating up and down. The front slide cavity 36 is provided with a front slide guide 62 on the lower wall. A front spring 25 is fixedly connected to the right end wall of the front slide cavity 36. The front spring 25 The left side is fixedly connected with a front sliding block 30 slidingly matched with the front sliding guide 62. The front end of the front sliding block 30 is fixedly connected with a front rope 31. A front hanging rod 28 is disposed above the front sliding block 30. The hanging rod 28 is fixedly connected to a front folding shed 29, and the other end of the front folding shed 29 is fixedly connected to the rear wall of the front sliding cavity 36. The front fixing rod 35 is fixed between the left and right walls of the front sliding cavity 36. The left and right sides of the rod 28 are fixedly installed on a winding block 32. The right side of the winding block 32 is wound with a front resetting rope 26 that spans the front fixing rod 35. The left and right winding blocks 32 are each connected to the front telescopic rod group. 33 is fixedly connected, and the left and right front telescopic rod groups 33 are connected to the front fixed rod through a connecting plate 34 35 sliding connection, the front support plate rear wall is fixedly connected with a front fixing plate 85 located on the left side of the front folding shed shrink box 24, the front fixing plate 85 is fixedly connected with a front through rope block 37 on the rear wall, and the front through A front left and right rope track 38 passes through the rope block 37 in the middle and left and right. The front left and right rope track 38 communicates with a front and back rope track 87 on the left and rear sides. The front and back rope track 87 and the front and left rope track 38 A front turning wheel 88 is slidably connected at the junction. The front turning wheel 88 is cooperatively connected with a front turning wheel axle 86 fixedly connected to the front rope block 37. The front rope 31 communicates with the front left and right through the front and rear through rope rails 87. The rope track 38 is fixedly connected to the left connecting rod 40 which is fixedly connected to the lower wall of the left moving block 4.

Beneficially, the right folding shed mechanism includes a right driving pulley 42 cooperatively connected with the right driving pulley shaft 58, the right driving pulley 42 meshes with the right timing belt 57, and the right timing belt 57 and the right moving block 16 meshing, the right moving block 16 is fixedly connected with a right clamping block 17 on the right wall, the right clamping block 17 is clamped and connected with the right folding shed 18, and the right folding shed 18 is folded and fixed on the right side of the car In the right folding shed shrink box 19 on the upper wall of the rear end, the right driven pulley shaft 83 is cooperatively connected with the right driven pulley 56, and the right driven pulley 56 is meshed with the right timing belt 57 for transmission.

Advantageously, the rear folding shed mechanism includes a rear folding shed shrink box 89 fixedly connected to the rear support plate 47, the rear folding shed shrink box 89 is provided with a rear sliding cavity 98 with an opening facing upward, and the rear folding shed shrinks The bottom wall of the box 89 is provided with a rear contraction groove 52 communicating up and down. A rear slide rail 53 is provided on the lower wall of the rear slide cavity 98. A rear spring 48 is fixedly connected to the left end wall of the rear slide cavity 98. A rear sliding block 51 that is slidably engaged with the rear sliding guide 53 is fixedly connected on the right side, and a rear rope 23 is fixedly connected to the right end surface of the rear sliding block 51. A rear droop rod 91 is disposed above the rear sliding block 51. The rear hanging bar 91 is fixedly connected to a rear folding shed 92, and the other end of the rear folding shed 92 is fixedly connected to a rear wall of a rear sliding cavity 98, and a rear fixing rod 27 is fixed between the left and right walls of the rear sliding cavity 98. The left and right sides of the rear drooping rod 91 are fixedly installed on the winding block 93. The winding block 93 on the left side is wound with a rear resetting rope 97 that spans the rear fixing rod 27. The left and right winding blocks 93 are each connected to the rear telescopic rod. Group 94 is fixedly connected, and the left and right rear telescopic rod groups 94 are connected to the rear through a connecting plate 95. The fixing rod 27 is slidingly connected. The front wall of the rear support plate is fixedly connected with a rear fixing plate 79 located on the right side of the rear folding shed shrink box 89, and the rear wall of the rear fixing plate 79 is fixedly connected with a rear through rope block 22. Rear left and right rope track 21 runs through the left and right rope track 21 in the middle and right and left and right through the rear left and right rope track 21, and there is a front and back rope track 81 communicating with the front and back. A rear turning wheel 82 is slidably connected at the junction of the track 21. The rear turning wheel 82 is mated with the rear turning wheel axle 80 fixedly connected to the rear rope block 22. The rear rope 23 is connected to the rear through the front and rear rope track 81. The left and right rope track 21 are fixedly connected to the right connecting rod 20, which is fixedly connected to the lower wall of the right moving block 16.

Advantageously, the front telescopic rod group 33 includes a plurality of front connecting rods 75, and the plurality of front connecting rods 75 are rotatably connected through a plurality of front circular hinges 76.

Advantageously, the rear telescopic rod group 94 includes a plurality of rear connecting rods 77, and the plurality of rear connecting rods 77 are rotatably connected through a plurality of rear circular hinges 78.

In the initial state, the top folding shed 11 is folded and shrunk in the top folding shed shrink box 9, the left folding shed 2 is folded and shrunk in the left folding shed shrink box 1, and the front folding shed 29 is folded and placed in front sliding On block 30, the left folding shed 18 is folded and shrunk in the left folding shed shrink box 19, the rear folding shed 92 is folded and placed on the rear sliding block 51, the front telescopic rod group 33 and the rear telescopic rod group 94 Both are in a contracted and folded state, and the front spring 25 and the rear spring 48 are both in a normal stretched state.

The sequence of mechanical actions of the entire device:

1: The starter motor 64 drives the front driving pulley shaft 67 to rotate, so that the front driving pulley 7 rotates, so that the front timing belt 10 meshing with the front driving pulley 7 is transmitted, so that the front meshing with the belt above the front timing belt 10 The moving block 12 moves to the right, so that the front clamping block 41 fixedly connected to the front moving block 12 moves to the right, so that the top folding shed 11 clamped and connected to the front clamping block 41 extends to the right, and the front driving belt The rotation of the wheel shaft 67 causes the front drive bevel gear 8 to rotate, so that the left drive bevel gear 6 meshing with the front drive bevel gear 8 rotates, so that the left drive pulley shaft 44 connected to the left drive bevel gear 6 rotates, so that The left driving pulley 5 connected with the driving pulley shaft 44 rotates, so that the left timing belt 68 meshing with the left driving pulley 5 is transmitted, so that the left moving block 4 meshing with the belt above the left timing belt 68 is moved backward, thereby The left clamping block 3 fixedly connected to the left moving block 4 is moved backward, so that the left folding shed 2 which is clamped and connected to the left clamping block 3 is extended backward, and the left timing belt 68 is driven to cause the left driven pulley 71 turns, so that the connected with the left driven pulley 71 The driven pulley shaft 45 is rotated, so that the left driven bevel gear 69 connected with the left driven pulley shaft 45 is rotated, so that the rear driven bevel gear 70 meshing with the left driven bevel gear 69 is rotated, and the rear driven The rear driving pulley shaft 72 connected with the bevel gear 70 is rotated to rotate the rear driving pulley 73 that is connected to the rear driving pulley shaft 72, so that the rear timing belt 43 meshing with the rear driving pulley 73 is transmitted, and The rear moving block 49 with the mesh above the rear timing belt 43 moves to the right, so that the rear clamping block 50 fixedly connected to the rear moving block 49 moves to the right, so that the top folding shed is tightly connected to the rear clamping block 50 11 stretches to the right, and the common movement of the front clamping block 41 and the rear clamping block 50 makes the extension of the ceiling 11 more stable. The front timing belt 10 drives the front driven pulley 14 to rotate, so that the front driven belt 14 is rotated. The front driven pulley shaft 59 connected with the wheel 14 is rotated, so that the front driven bevel gear 13 connected with the front driven pulley shaft 59 is rotated, so that the right driven bevel gear 15 meshed with the front driven bevel gear 13 is rotated. So that the right connected with the right driving bevel gear 15 The moving pulley shaft 58 rotates, so that the right driving pulley 42 connected with the right driving pulley shaft 58 rotates, so that the right timing belt 57 meshing with the right driving pulley 42 is transmitted, thereby meshing with the upper belt of the right timing belt 57 The right moving block 16 moves forward, so that the right clamping block 17 fixedly connected to the right moving block 16 moves forward, so that the right folding shed 18 clamped and connected to the right clamping block 17 extends forward;

2: The left moving block 4 moves backward, so that the left connecting rod 40 fixedly connected to the lower wall of the left moving block 4 moves backward, so that the front rope 31 fixedly connected to the left connecting rod 40 passes through the front and rear rope pass rails. 87 and the front left and right rope track 38 are moved backward, so that the front slide block 30 fixedly connected to the front rope 31 slides to the left on the front slide guide 62, so that the front hanging bar 28 disposed on the front slide block 30 is on itself Falling through the front shrinking groove 61 under the action of gravity, so that the front folding shed 29 extends downward;

3: The right moving block 16 moves forward, so that the right connecting rod 20 fixedly connected to the lower wall of the right moving block 16 moves forward, so that the rear rope 23 fixedly connected to the right connecting rod 20 passes through the rear front and rear rope track. 81 and the rear left and right rope track 21 are moved forward, so that the rear slide block 51 fixedly connected to the rear rope 23 slides to the right on the rear slide guide 53 so that the rear droop rod 91 disposed on the rear slide block 51 is on itself Falling through the rear contraction groove 52 under the action of gravity, so that the rear folding shed 92 extends downward;

4: When the device needs to be reset, the motor 64 drives the front active pulley shaft 67 to reverse, so that the front active pulley 7 is reversed, and the front timing belt 10 is reversely driven, so that the front moving block engaging the belt above the front timing belt 10 12 moves to the left, so that the front clamping block 41 fixedly connected to the front moving block 12 moves to the left, so that the top folding shed 11 clamped and connected to the front clamping block 41 shrinks to the left and returns to the top folding shed shrink box 9 Inside, the front drive pulley shaft 67 is reversed to reverse the front drive bevel gear 8 so that the left drive bevel gear 6 meshing with the front drive bevel gear 8 is reversed, so that the left drive belt that is cooperatively connected with the left drive bevel gear 6 The axle 44 is reversed, so that the left driving pulley 5 connected to the left driving pulley shaft 44 is reversed, so that the left timing belt 68 meshing with the left driving pulley 5 is reversely driven, so that the left timing belt 68 is above the left timing belt 68. The meshed left moving block 4 moves forward, so that the left clamping block 3 fixedly connected to the left moving block 4 moves forward, so that the left folding shed 2 clamped and connected to the left clamping block 3 retracts forward. In the left folding shed contraction box 1, the left timing belt 68 reversely drives the left driven pulley 71 Therefore, the left driven pulley shaft 45 that is cooperatively connected with the left driven pulley 71 is reversed, and the left driven bevel gear 69 that is cooperatively connected with the left driven pulley shaft 45 is reversed, so that the left driven cone The rear driving bevel gear 70 meshed with the gear 69 is reversed, thereby reversing the rear driving pulley shaft 72 cooperatively connected with the rear driving bevel gear 70, thereby reversing the rear driving pulley 73 cooperatively connected with the rear driving pulley shaft 72, Thus, the rear timing belt 43 meshing with the rear driving pulley 73 is reversely driven, so that the rear moving block 49 meshing with the belt above the rear timing belt 43 is moved to the left, so that the rear clamp fixedly connected to the rear moving block 49 is clamped. The block 50 is moved to the left, so that the top folding shed 11 clamped and connected to the rear clamping block 50 is contracted to the left back to the top folding shed shrink box 9, and the common movement of the front clamping block 41 and the rear clamping block 50 makes the ceiling The contraction of 11 is more stable. The reverse transmission of the front timing belt 10 reverses the front driven pulley 14, thereby reversing the front driven pulley shaft 59 which is cooperatively connected with the front driven pulley 14. The front driven bevel gear 13 coupled with the driving pulley shaft 59 is reversed, so that the front driven bevel gear 13 is connected with the front driven bevel gear 13 The closed right driving bevel gear 15 is reversed, so that the right driving pulley shaft 58 connected to the right driving bevel gear 15 is reversed, so that the right driving pulley 42 connected to the right driving pulley shaft 58 is reversed, so that The right timing belt 57 meshed with the right driving pulley 42 is reversely driven, so that the right moving block 16 meshing with the belt above the right timing belt 57 is moved backward, so that the right clamping block 17 fixedly connected to the right moving block 16 is moved. Move backward so that the right folding shed 18 clamped and connected to the right clamping block 17 retracts back to the right folding shed shrink box 19, manually pull down the front resetting rope 26 to shrink the front telescopic rod group 33, and the left moving block 4 Moving forward loosens the front rope 31, so that the stretched front spring 25 is reset, so that the front droop rod 28 is resettled on the front sliding block 30, and the rear reset rope 97 is manually pulled down to contract the rear telescopic rod group 94, right The moving block 16 moves backward to relax the rear rope 23, thereby resetting the stretched rear spring 48, so that the rear drooping rod 91 is resettled on the rear sliding block 51, and the device is reset.

The above embodiments are only for explaining the technical concept and characteristics of the present invention, and the purpose thereof is to enable those skilled in the art to understand and implement the content of the present invention, but not to limit the protection scope of the present invention. Any equivalent change or modification made according to the spirit and essence of the present invention should be covered by the protection scope of the present invention.

1. A detector capable of autonomously detecting the oral cavity includes a headband, which is characterized in that a detector is fixedly arranged at the right end of the headband;
an eyecup is fixed on the left end surface of the detector. After the headband is worn on the head, the eyecup is placed around the eyes. A display screen is provided in the headband and on the right side of the eyecup. A light passage is provided between the display screen and the eyecup, and a light blocking plate is provided in the light passage;
an opening and closing device is provided on the upper side of the display screen and the light-passing port, and a lifting plate is provided in the opening and closing device. The left end of the lifting plate is fixedly connected to the upper end of the right end face of the light blocking plate. Drive the light blocking plate up and down and open and close the light passage;
a storage cavity is provided on the right end of the lower end face of the headband, a camera is installed in the storage cavity, a winding device is provided on the upper side of the storage cavity, and a winding rod is provided in the winding device. A transmission line is wound on the rod, the lower end of the transmission line is fixed and electrically connected to the upper end of the camera, the other end is fixed and electrically connected to the right end of the display screen, and the winding rod is dynamically connected to the lifting plate;
the transmission line is elongated by manual pulling, and then the winding rod is driven to rotate, which in turn drives the lifting plate and the light blocking plate to rise and open the light passing port, and the camera is manually extended into the cavity for shooting detection. The video data is transmitted to the display screen through the transmission line for display, and then the oral cavity is observed by means of VR.

2. The detector capable of autonomously detecting the oral cavity according to claim 1, wherein the opening and closing device comprises a baffle cavity connected to an upper wall of the light passage opening, and the light blocking plate is slidably arranged up and down. Inside the baffle cavity, a right-hand wall of the baffle cavity is provided with a lifting cavity, the lifting plate is slidably provided in the lifting cavity, and a rotatable between the upper and lower walls of the lifting cavity is provided. A lifting screw screwed to the lifting plate, a bevel gear cavity is provided on the lower side of the lifting cavity, and the lower end of the lifting screw extends into the bevel gear cavity to be fixed with a driven bevel gear. A driving bevel gear which is rotatably provided on the wall is engaged with the driven bevel gear, and the driving bevel gear is fixedly provided with a rotating shaft at its axis.

3. The detector capable of autonomously detecting the oral cavity according to claim 1, wherein the winding device comprises a winding cavity provided on an upper side of the storage cavity, and the winding cavity and the storage cavity A communication port is provided between the winding rods, and the winding rod is rotatably connected between the front and rear walls of the winding cavity. One end of the transmission line passes through the opening and is fixedly connected to the camera. One end passes through the left wall of the winding cavity and is fixedly connected to the display screen. The outer surface of the winding rod is symmetrical and fixed with a turntable on the front and back sides of the transmission line. The array has beveled card slots.

4. The detector capable of autonomously detecting the oral cavity according to claim 3, wherein a torsion spring is fixedly connected between an end of the turntable far from the center of symmetry and the inner wall of the winding cavity, and the torsion spring can be twisted by The force drives the turntable and the winding rod to turn over and winds and stores the transmission line.

5. The detector capable of autonomously detecting the oral cavity according to claim 3, wherein a spur gear cavity located behind the bevel gear cavity is provided on the rear side of the winding cavity, and behind the winding rod, A driving spur gear is fixed to the spur gear cavity at one end, and a driven spur gear is fixed to the left end of the driving spur gear at the rear end of the rotating shaft.

6. A detector capable of autonomously detecting the oral cavity according to claim 3, wherein a lower pressure chamber is provided on the upper side of the winding cavity, and a lower pressure plate is provided in the lower pressure chamber so as to be able to slide up and down. An opening slot is provided between the lower pressing cavity and the winding cavity. A lower end of the lower pressing plate is fixedly provided with a card plate that passes through the opening slot and extends into the winding cavity. The lower end of the card plate It can be extended into the inclined grooves on the front and back sides to lock and lock the turntable.

7. The detector according to claim 6, wherein a lower pressing spring is fixedly connected between the upper end of the lower pressing plate and the upper wall of the lower pressing cavity.

8. The detector capable of autonomously detecting the oral cavity according to claim 6, characterized in that a lock lever slide cavity is provided between the left wall of the lower pressure cavity and the right wall of the lifting cavity, and the lock lever slides There are lock levers that can slide left and right in the cavity. The left and right ends of the lock lever are provided with inclined surfaces, the left side is inclined downward, and the right side is inclined upward. A pressing cavity is communicated with the upper wall of the lever sliding cavity, and a pressing plate is slidably arranged in the pressing cavity. The pressing plate penetrates and is fixed in the pressing plate, and the lower end of the pressing rod can extend to the concave A push button is fixed in the groove to push the lock lever to the right, and the upper end of the pressing lever extends to the upper side of the detector.

9. The detector according to claim 8, wherein a tension spring is fixedly connected between the upper end of the pressing plate and the upper wall of the pressing cavity.
